(19) United States
(12) Patent Application Publication
(10) Pub. No.: US 2020/0214636 A1
(43) Pub. Date: Jul. 9, 2020

(54) BACTERIA SAMPLING AND DISPERSION CAPSULE

(71) Applicant: CHECK-CAP LTD., Isfiya (IL)

(72) Inventors: Yoav KIMCHY, Haifa (IL); Shlomo LEWKOWIZ, Kiryat Tivon (IL); Sigalit KIMCHY, Haifa (IL)

(21) Appl. No.: 16/627,367

(22) PCT Filed: Jun. 7, 2018
(86) PCT No.: PCT/IL2018/050623

§ 371 Date: Dec. 30, 2019

Related U.S. Application Data
(60) Provisional application No. 62/528,157, filed on Jul. 3, 2017.

(51) Int. Cl. A61B 5/00; A61B 5/03; A61B 5/07; A61B 10/00; A61B 90/00

(52) U.S. Cl. CPC A61B 5/6861; A61B 5/036; A61B 5/073; A61B 5/42; A61B 2562/162; A61B 10/0045; A61B 90/36; A61B 2010/0061; A61B 2562/0247; A61B 5/4839

(57) ABSTRACT

A capsule for traversing the gastrointestinal tract, including one or more compartments configured to sample the environment surrounding the capsule or configured to deliver content to the environment surrounding the capsule, an internal controller configured to activate the one or more compartments to sample or deliver, wherein the capsule includes a tracking system configured to identify the location of the capsule in real-time and activate a compartment when reaching a specific location in the gastrointestinal tract.

### RELATED APPLICATIONS

The present application claims priority from U.S. Provisional application No. 62/528,157 filed on Jul. 3, 2017, the disclosure of which is incorporated herein by reference.

### TECHNICAL FIELD

The present disclosure relates generally to the collection of bacterial samples from different locations along the length of the gastrointestinal track and the dispersion of bacteria to specific locations along the length of the gastrointestinal track.

### BACKGROUND

The role of bacteria colonies in the colon in keeping the hosting individual healthy is being investigated in recent years. There appears to be a high correlation between a well-balanced and diverse population of the right type of bacteria in the colon and good health. Additionally, patients with low bacteria diversity and imbalance of bacteria colonies tend to be more prone to illness such as Irritable Bowel Disease (IBD), Colitis and Crones disease (CD). These chronic inflammatory bowel syndromes are correlated to the bacteria population in the small bowel and colon but the exact mechanism and influence of these bacteria colonies and bacteria species on the health of the individual is not well understood. A major reason for this lack of understanding relates to the absence of an accurate and repeatable method for sampling the bacterial contents of the small bowel and probably more importantly sampling in the colon.

New experimental treatments are trying to improve the diversity of colon bacteria by implanting stool from healthy individuals into colon of patients with certain bacterial deficiencies. It may also be beneficial to use a more precise method and introduce certain types of bacteria to specific locations in the colon to enhance the colon bacteria population and perhaps improve the health of patients by this select and localized bacteria implant. Additionally, the practice of stool “implantation” may be more beneficial if the “implanted stool” will be done in precise location in the colon where it is determined to be most beneficial to the patient without cleansing the colon for colonoscopy insertion.

It is therefore desirable to selectively sample colon contents containing bacteria from different areas along the length of the colon in order to learn about the distribution of bacteria in the colon in a way that is patient friendly and reliable without the need for an invasive procedure and with minimal discomfort to the patient. In addition, it is advantageous to collect such localized bacteria samples from the small bowel or the colon in its natural state rather than with a colonoscope that requires bowel cleansing that may distort the data.

Additionally, localized specific administration of bacteria or dispensing specific antibiotics at precise locations may enable to change the bacteria of the small bowel or colon in a way that will benefit the patient.

### SUMMARY

An aspect of an embodiment of the disclosure relates to a system and method for dealing with the colon and/or the entire gastrointestinal tract of a user. The system includes a capsule with one or more compartments. Each compartment may include a sampling system to sample bacteria surrounding the location of the capsule or a delivery system to deliver content to the location surrounding the capsule. The sampling system may include a vacuum to suck in content from the environment surrounding the capsule when instructed to collect a sample by an internal controller of the capsule. The sampling system may store the content in the compartment so that it may be analyzed by retrieving the capsule after it exits the user. Alternatively, the sampling system may analyze the content in real-time and provide the results to the internal controller of the capsule. The delivery system may receive instructions from the internal controller to deliver a dose of medication or bacteria to the location surrounding the capsule.

Additionally, the capsule includes a tracking system that may cooperate with an external controller to identify the location of the capsule so that the internal controller can activate the compartments responsive to reaching a pre-determined location. In some embodiments of the disclosure, an imaging capsule is used as a scout before using the capsule to identify locations of interest for sampling or delivering. Optionally the external controller may record the locations of interest and then notify the capsule of the locations of interest so that the capsule can activate compartments at the locations of interest.

There is thus provided according to an exemplary embodiment of the disclosure, a capsule for traversing the gastrointestinal tract, comprising:

One or more compartments configured to sample the environment surrounding the capsule or configured to deliver content to the environment surrounding the capsule;

An internal controller configured to activate the one or more compartments to sample or deliver;

A tracking system configured to identify the location of the capsule;

Wherein the internal controller activates a compartment responsive to the location of the capsule in the gastrointestinal tract.

In an exemplary embodiment of the disclosure, the internal controller activates compartments responsive to instructions from an external controller. Alternatively or additionally, the internal controller is configured to activate compartments responsive to sampling results received from another compartment that was previously activated. In an exemplary embodiment of the disclosure, the internal controller activates multiple compartments simultaneously. Optionally, the internal controller receives information from compartments that sample the environment relating to the type and/or count of bacteria surrounding the compartment. In an exemplary embodiment of the disclosure, all of the compartments are sampling compartments or all of the compartments are delivery compartments. Alternatively, some of the compartments are sampling compartments and some are delivery compartments. In an exemplary embodiment of the disclosure, the capsule is protected by a shell with shell slots to serve as inlets or outlets for the capsule, wherein the compartments have compartment slots to serve as inlets or outlets from the compartments, and wherein the compartments are rotatable relative to the shell and are activated by aligning the compartment slot with the shell slot. Optionally, the capsule includes an imaging system and activates compartments responsive to analyzing images in real-time. In an exemplary embodiment of the disclosure, the capsule includes a pressure sensor and activates compartments responsive to pressure levels inside the capsule. Optionally, the capsule includes electrodes on a shell of the capsule and the electrodes are charged when delivering medication to induce electrophoresis. In an exemplary embodiment of the disclosure, the one or more compartments are configured to be activated to sample or deliver multiple times. Optionally, the one or more compartments include a piston to push content out of the compartment. In an exemplary embodiment of the disclosure, the one or more compartments are initialized with a vacuum to pull in content for sampling.

There is further provided according to an exemplary embodiment of the disclosure, a method of sampling the gastrointestinal tract or delivering content to the gastrointestinal tract, comprising:

Ingesting a capsule with one or more compartments configured to sample the environment surrounding the capsule or configured to deliver content to the environment surrounding the capsule;

Tracking the location of the capsule in real-time with a tracking system configured to identify the location of the capsule;

Notifying an internal controller with the identified location of the capsule;

Activating the one or more compartments to sample or deliver responsive to the location in the gastrointestinal tract.

### BRIEF DESCRIPTION OF THE DRAWINGS

The present disclosure will be understood and better appreciated from the following detailed description taken in conjunction with the drawings. Identical structures, elements or parts, which appear in more than one figure, are generally labeled with the same or similar number in all the figures in which they appear, wherein:

FIG. 1 is a schematic illustration of a system for dealing with a colon, according to an exemplary embodiment of the disclosure;

FIG. 2 is a schematic illustration of a capsule with multiple compartments, according to an exemplary embodiment of the disclosure;

FIG. 3 is a schematic illustration of a capsule with a single compartment, according to an exemplary embodiment of the disclosure; and

FIG. 4 is a flow diagram of a method of treating a colon, according to an exemplary embodiment of the disclosure.

### DETAILED DESCRIPTION

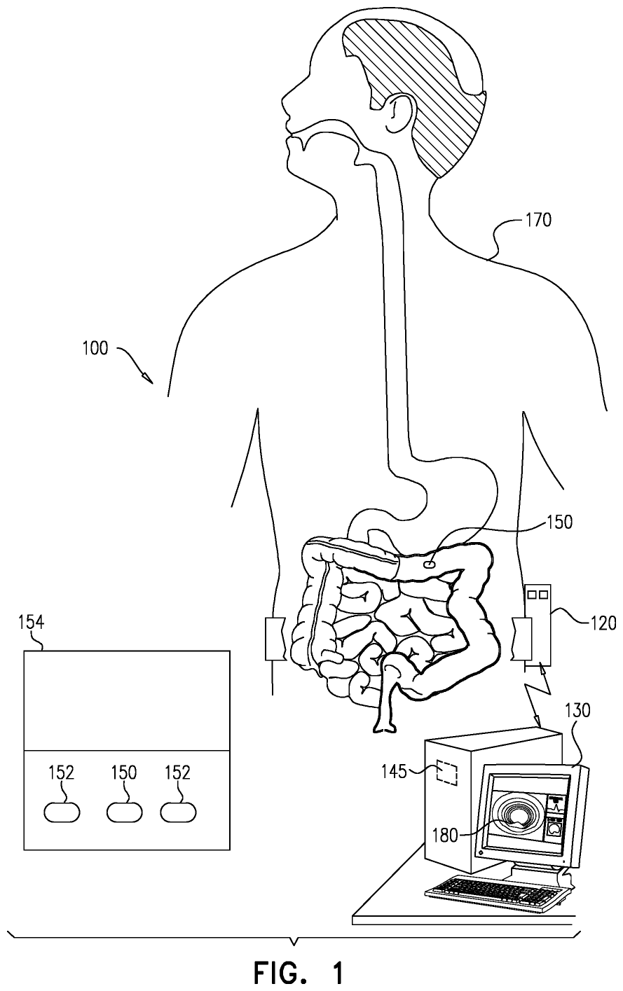

FIG. 1 is a schematic illustration of a system 100 for dealing with a colon 110, according to an exemplary embodiment of the disclosure. System 100 includes a capsule 150 that is ingested by a user 170 to traverse the user's gastrointestinal tract (including the colon 110) from the inside. In an exemplary embodiment of the disclosure, the capsule 150 identifies its location with a tracking system. Optionally, the tracking system may identify its location independently or with the aid of an external controller 120. In some embodiments of the disclosure capsule 150 may also use additional information to verify its location (e.g. based on time, pressure, distance traveled). Optionally, capsule 150 is configured to sample bacteria at specific locations and/or deliver medication or bacteria at the specific locations.

In an exemplary embodiment of the disclosure, an imaging capsule 152 that uses optics and/or radiation to produce images of colon 110 of the user may be used prior to using capsule 150 to map out the colon 110 and identify locations of interest for delivering or sampling. Optionally, the user may be provided with a kit 154 comprising imaging capsule 152 and capsule 150 to map the colon and then deliver medication or bacteria to previously identified locations. Alternatively or additionally, capsule 150 may include an imaging system and may determine where to sample or deliver in real time as it traverses the colon 110. In some embodiments of the disclosure, kit 154 may include three capsules: an imaging capsule 152 for pre-delivery scouting of colon 110, a capsule 150 for delivering and/or sampling and a post imaging capsule 152 to check the results of treating the colon 110. Alternatively or additionally, the kit may include two capsules 150, one to sample areas of the colon to identify bacteria and a second capsule 150 to deliver medication based on the results of the first capsule 150.

In an exemplary embodiment of the disclosure, while traveling inside the gastrointestinal tract of user 170, capsule 150 communicates with an external controller 120 that receives information from the capsule 150 and provides instructions and information to the capsule 150, based on the location of capsule 150 and other information provided by capsule 150. Optionally, external controller 120 may communicate with a computer 130 that executes an analysis application 145 to analyze the information received from the capsule 150 and provide information to the capsule 150. For example computer 130 may receive images or location information, which may be analyzed to identify the current location of the capsule 150 and determine if the capsule 150 should perform any actions. Optionally, computer 130 may provide or display images 180 of the inside of the colon 110 to a practitioner dealing with user 170. In an exemplary embodiment of the disclosure, program 145 can be stored on a non-volatile computer storage medium (e.g. CD, DVD, USB drive) to be transferred to computers to analyze scan data and reconstruct images of a colon or a segment of a colon.

In an exemplary embodiment of the disclosure, capsule 150 may be tracked as it traverses the gastrointestinal tract of the user 170 as described for example in U.S. patent application Ser. No. 14/785,860 (published as US 2016/0066813) the disclosure of which is incorporated herein by reference. Alternatively, other tracking methods may be used to track the capsule 150 in 3D and identify the location of the capsule 150 in real-time as it flows through the gastrointestinal tract of the user 170. In some embodiments of the disclosure, capsule 150 may determine its location based on cooperation with an external controller 120.

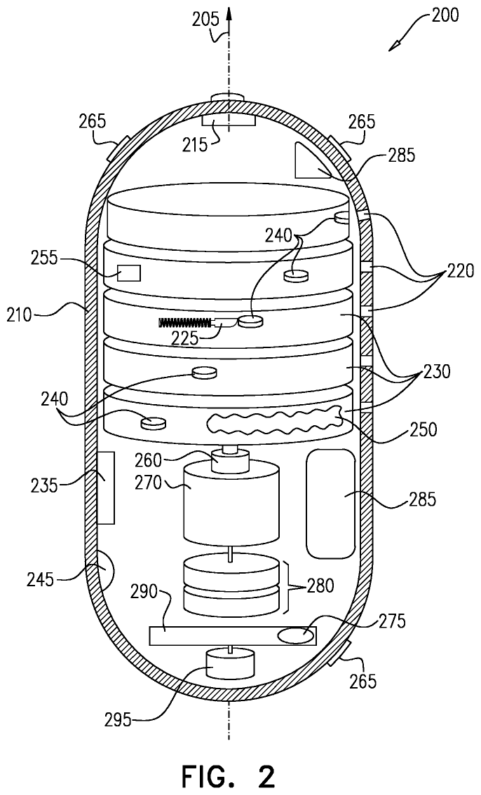

FIG. 2 is a schematic illustration of a capsule 200 with multiple rotatable compartments 230, according to an exemplary embodiment of the disclosure. The capsule shell 210 is typically made of polycarbonate or other materials that can withstand the pressure and fluids inside the body of the user 170. Optionally, the capsule shell 210 comprises one or more rotatable compartments 230 for sampling bacteria or delivering medication or bacteria. Each compartment comes with a compartment slot 240 that serves as an inlet or outlet for absorbing content or releasing content. Likewise the capsule shell 210 comprises shell slots 220 that serve as inlets or outlets for the rotatable compartments 230 from the capsule shell 210. Optionally, when the compartment slot 240 is aligned with the shell slot 220 the compartment 230 can be activated to release or absorb content.

Initially the compartment slots 240 are all blocked by capsule shell 210. Optionally, each compartment 230 may be rotated independently or all the compartments 230 may be rotated together. The shell slots 220 may all be aligned on a side of the shell 210 parallel to elongated axis 205, whereas the compartment slots 240 may be displaced relative to each other, for example (diagonally or not diagonally) so that as the compartments 230 rotate together, the compartments 230 will be activated in turn (to release or absorb) sequentially or not sequentially (e.g. from top to bottom, bottom to top or any other order). Alternatively, the shell slots 220 may be arranged around the circumference of the capsule shell 210 and the compartment slots 240 aligned axially one on top of each other.

In an exemplary embodiment of the disclosure, each compartment 230 may initially maintain a vacuum 250 so that when compartment slot 240 is aligned with shell slot 220, content from outside the capsule 200 is sucked into the compartment 230. Alternatively compartment 230 may contain a payload to be released by the compartment 230, for example the payload may contain a dose of an antibiotic drug to target specific bacteria populations or a bacteria to be released in the small bowel or colon, or the payload may contain donor feces to be released at specific location in the colon 110, Optionally, the payload may be held under pressure so that it will burst out when the compartment slot 240 and shell slot 220 are aligned.

In an exemplary embodiment of the disclosure, each compartment 230 may be activated at a different location along the colon 110, for example at pre-selected locations. In an exemplary embodiment of the disclosure, capsule 200 includes a motor 270 and a gear 260 to rotate the compartments 230. Optionally, when a compartment is aligned it enables and/or activates the designed function of the compartment, being either sampling the surrounding environment or releasing a payload to the environment. In an exemplary embodiment of the disclosure, capsule 200 may include a power source such as a battery 280, an imaging system 215 (e.g. a camera to view the surroundings), an internal electronic controller circuit 290, an RF transceiver 275, an electronic localization system 295 (e.g. with a coil) and one or more electrodes 265 for performing electrolysis.

In an exemplary embodiment of the disclosure, external controller 120 tracks the location of capsule 200 based on the readings from the electronic localization system 295. Optionally, controller 290 communicates via RF transceiver 275 with external controller 120 to instruct controller 290 to activate a compartment 230. External controller 120 or computer 130 may be programmed to remember the actions/mappings of all capsules 150 (or imaging capsules 152) ingested by the user 170. Accordingly, external controller 120 may instruct multiple capsules to sample or release at different locations in the colon 110, for example to prevent repetitively treating the same location.

In an exemplary embodiment of the disclosure, compartments 230 may be activated at an absolute location, periodically, or based on a specific distance of advancing in the colon 110 (e.g. travel length) or at specific predesignated locations. Likewise the compartments 230 may be activated after a specific time interval (e.g. after six hours or when entering a specific organ (e.g. at the Cecum, which is the beginning of the colon 110).

In an exemplary embodiment of the disclosure, capsule 150 is manufactured with a specific density greater than 1 gram/cc (water) or even greater than 2 gram/cc so that the capsule will sink and lie on the colon lumen surface while traveling or staying in the colon. Optionally, shell slots 220 are positioned along the length of capsule 200 and capsule 200 is designed to have an asymmetrical weight distribution around its axis (e.g. with the help of weights 285) so that the capsule 200 tends to rotate and settle with the sample collecting holes (shell slots 220) of the outer capsule shell facing and possibly touching the colon mucosal surface. This arrangement enhances the ability to collect bacteria on the colon mucosal surface while traversing the colon 110. Optionally, the weights 285 in capsule 200 are positioned inside capsule shell 210 on the side of shell slots 220. Alternatively, weights 285 may be embedded in the capsule shell 210.

Collection from the small bowel of the user 170 is generally always possible from the lumen surface since the capsule's diameter is designed to touch the small bowel all the time, having a capsule diameter ranging typically between about 8 mm to 13 mm.

In an exemplary embodiment of the disclosure, some of the compartments 230 may be manufactured to sample the surrounding environment and some may be designed to release a substance to the surrounding environment. For example the compartments 230 may be arranged so that they alternately sample or release (e.g. a first compartment 230 may sample and the second adjacent compartment 230 may release and so forth). Alternatively, specific arrangements may be used such as the first and last compartment 230 may sample and the rest may release or vice versa.

In an exemplary embodiment of the disclosure, when releasing medication controller 290 may activate electrodes 265 to create electrophoresis to enhance the kinetic properties of the medication and enhance transfer of the medication to the blood stream.

In an exemplary embodiment of the disclosure, compartments 230 that serve as sampling compartments may contain a soaking material that will absorb the sampled content and keep it mechanically stable for later analysis. In another aspect of this invention, a “stabilizing material” in the sampling compartments is used to preserve the bacteria sampled for later analysis. Such a stabilizing material can be RNAlater, OMNIgene.GUT, Tris-EDTA among others.

In an exemplary embodiment of the disclosure, amplicon sequencing targeted to the 16S ribosomal RNA gene is used to identify the bacteria strains in the samples. Optionally, a DNA micro array chip (lab on a chip) is placed in the sampling chambers and once a sample is collected, the chip senses the 16S ribosomal RNA gene or other gene type to determine the type of bacteria and the relative number of each bacteria strain in the sample.

Optionally, the data from the lab on chip is then collected by the capsule controller 290 and sent via RF transceiver 275 over an RF channel to the external controller 120. In this way, the capsule 150 is not required to be collected by the user 170 as all the data is transmitted from the capsule 150 to the externally worn external controller 120.

In an exemplary embodiment of the disclosure, different compartments 230 may hold different types of medication. Optionally, responsive to the results from sampling bacteria with one of the compartments 230, controller 290 may select a specific compartment 230 to release a specific medication to deal with the identified bacteria at or near the sampling location. Accordingly, during the journey of capsule 150 some of the compartments 230 may be activated and some of the compartments 230 may remain unused.

In an exemplary embodiment of the disclosure, the sampling compartments 230 have a small scooping tool 225 that scoops a small amount of the colon tissue as well as some of the mucosa that is closely bound to the colon tissue and contains a big portion of the bacteria of interest.

In an exemplary embodiment of the disclosure, a pressure sensor 245 is built into the capsule and measures the internal pressure of the capsule. When the capsule enters the colon 110, fermentation by bacteria in the colon 110, and especially at the beginning of the colon 110 in the Cecum releases Hydrogen gas, SO2, Methane, CO2 and other gases, some of which diffuse into the capsule through the capsule shell 210 and increase the internal pressure, this in turn gives an indication that the capsule has reached the colon 110. In an exemplary embodiment of the disclosure, the capsule shell 210 has parts that are membrane permeable to gases but not to liquids. This allows gas to enter the capsule faster and gives a faster indication of when the capsule 150 enters the colon 110.

In an exemplary embodiment of the disclosure, a micro machined gas chromatograph 235 is located in the capsule to analyze and identify the gases that enter the capsule. The identity of the gases is provided to the controller 290, which may relay the information to the external controller 120.

In an exemplary embodiment of the disclosure, an FIT (Fecal Immunologic Test) 255 for detecting the presence of blood is embedded in one or more of the sampling compartments and optics to read this test are embedded in the capsule 200 providing the results to controller 290. The Fecal Immunologic Test 255 allows sampling of colon contents at any desired point according to the capsule position, analyzing the Fecal Immunologic test 255 results in the capsule and sending the result via RF transceiver 275 to the external controller 120.

In an exemplary embodiment of the disclosure, a compartment 230 that serves as a dispenser is used to release/deliver/disperse bacteria, antibiotics, implant stool from healthy donors or other material at specific locations in the colon 110 or the gastrointestinal tract.

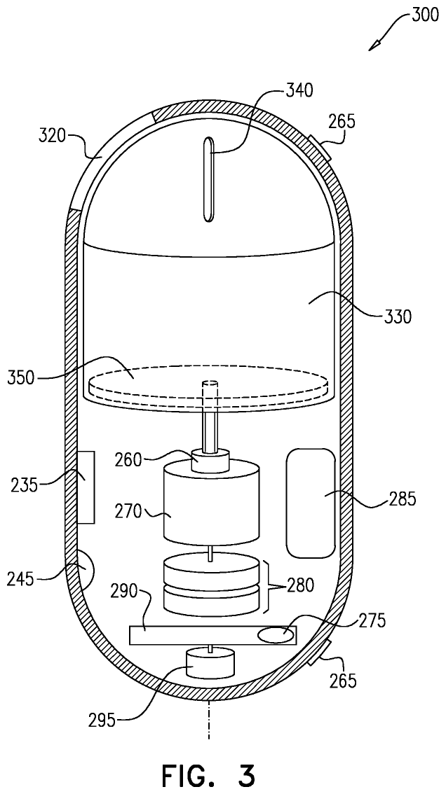

FIG. 3 is a schematic illustration of a capsule 300 with a single compartment 330, according to an exemplary embodiment of the disclosure. In an exemplary embodiment of the disclosure, capsule 300 is similar to capsule 200 however capsule 300 is provided with a single compartment 330 instead of multiple compartments 230. Optionally, the single compartment 330 is larger than compartments 230 and enables delivery of a single large load or taking a large sample. In an exemplary embodiment of the disclosure, capsule 300 samples or delivers at a single position in colon 110. Alternatively, compartment 330 may be activated and deactivated multiple times at different locations in the gastrointestinal tract of the user 170, for example to collect samples at multiple locations into a single compartment 330 or deliver from a single compartment 330 at multiple locations.

In an exemplary embodiment of the disclosure, compartment 330 is equipped with a compartment slot 340 that aligns with a matching shell slot 320 to activate the compartment. Optionally, compartment slot 340 and shell slot 320 are larger than compartment slot 240 and shell slot 220 to enable sampling or releasing larger items, for example small solid grains of a substance, instead of liquids and gases. In an exemplary embodiment of the disclosure, compartment 330 includes a piston 350 for pushing the content of the compartment 330 out from shell slot 320 via compartment slot 340. Optionally, capsule 300 includes similar elements as capsule 200, such as controller 290, motor 270 and gear 260. Capsule 300 utilizes motor 270 and gear 260 to control rotation of compartment 340 and pushing or pulling of piston 350 responsive to commands given by controller 290 of capsule 300.

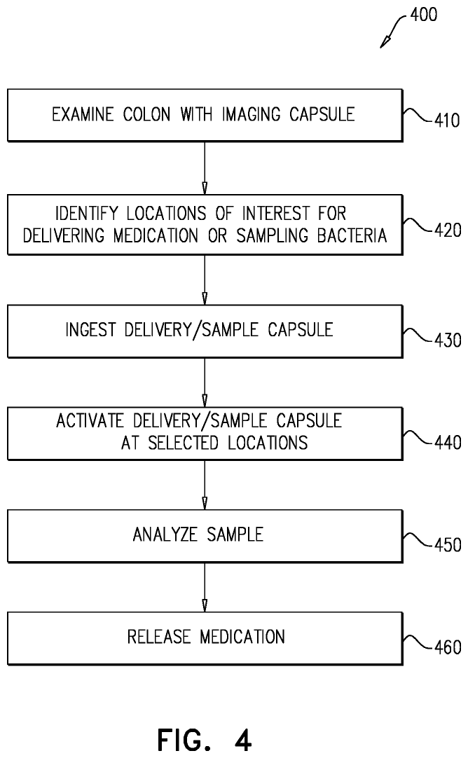

FIG. 4 is a flow diagram of a method 400 of treating colon 110, according to an exemplary embodiment of the disclosure. In an exemplary embodiment of the disclosure, user 170 may ingest imaging capsule 152 to examine (410) the user's gastrointestinal tract, especially the colon 110. Optionally, the imaging capsule records images of the journey, typically identifying (420) problematic locations, for example having polyps or other problems. The information is generally stored at computer 130 and/or at external controller 120. A practitioner may determine from the images that certain locations should be examined by sampling bacteria to identify more specifically the content of the colon 110 at those locations. Alternatively or additionally, user 170 may have symptoms, which the practitioner is interested in examining and may use capsule 150 to sample bacteria and report if specific types of bacteria exist in colon 110 of the user 170. Further alternatively or additionally, a practitioner may be interested in delivering bacteria or medication to a specific location in the colon 110 (e.g. the Cecum) to treat the user 170 based on information about the user 170 from other sources.

In an exemplary embodiment of the disclosure, the user 170 ingests (430) a capsule 150 to sample or deliver bacteria to the colon 110. Optionally, capsule 150 is tracked by a tracking system to identify the location of the capsule 150 and decide if to sample the surroundings or deliver a payload. Alternatively or additionally, capsule 150 may use other indications to determine if it is at the correct location to take actions. For example based on readings of a pressure sensor, based on an elapsed time, based on a distance traveled, based on results of sampling the surrounding environment.

In an exemplary embodiment of the disclosure, capsule 150 may activate (440) one or more compartments 230 in the capsule 150 to sample the environment to identify if specific types of bacteria are present, or to deliver bacteria, medication or other substances. Optionally, when sampling the environment the sample may be analyzed (450) immediately by the capsule 150 or may be stored in the capsule and analyzed after the capsule exits from the user 170. In an exemplary embodiment of the disclosure, the capsule 150 or external controller 120 may provide a warning when the capsule 150 is approaching the exit from the user 170.

In an exemplary embodiment of the disclosure, capsule 150 may release medication (460) either based on pre-programmed instructions or based on the results of analyzing samples in real-time. In some embodiments of the disclosure, capsule 150 may include imaging system 215 to view and analyze the current location and determine if to activate a compartment 230 at the current location. Optionally, controller 290 may activate two or more compartments 230 simultaneously, for example to compare the results of two sampling compartments 230 or to sample while delivering a bacteria.

Alternatively or additionally, additional capsules 150 or imaging capsules 152 may be used responsive to use of a previous capsule, for example to provide medication based on previous sampling results, to verify the effectiveness of treatment (visually or by sampling), to provide additional doses of medication or other reasons.

It should be appreciated that the above described methods and apparatus may be varied in many ways, including omitting or adding steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure.

It will be appreciated by persons skilled in the art that the present disclosure is defined by the claims and not limited to what has been particularly shown and described hereinabove.

I/We claim:

1. A capsule for traversing the gastrointestinal tract, comprising:
one or more compartments configured to sample the environment surrounding the capsule or configured to deliver content to the environment surrounding the capsule;
an internal controller configured to activate the one or more compartments to sample or deliver;
a tracking system configured to identify the location of the capsule;
wherein the internal controller activates a compartment responsive to the location of the capsule in the gastrointestinal tract.

2. The capsule of claim 1, wherein the internal controller activates compartments responsive to instructions from an external controller.

3. The capsule of claim 1, wherein the internal controller is configured to activate compartments responsive to sampling results received from another compartment that was previously activated.

4. The capsule of claim 1, wherein the internal controller activates multiple compartments simultaneously.

5. The capsule of claim 1, wherein the internal controller receives information from compartments that sample the environment relating to the type and/or count of bacteria surrounding the compartment.

6. The capsule of claim 1, wherein all of the compartments are sampling compartments or all of the compartments are delivery compartments.

7. The capsule of claim 1, wherein some of the compartments are sampling compartments and some are delivery compartments.

8. The capsule of claim 1, wherein the capsule is protected by a shell with shell slots to serve as inlets or outlets for the capsule, wherein the compartments have compartment slots to serve as inlets or outlets from the compartments, and wherein the compartments are rotatable relative to the shell and are activated by aligning the compartment slot with the shell slot.

9. The capsule of claim 1, wherein the capsule includes an imaging system and activates compartments responsive to analyzing images in real-time.

10. The capsule of claim 1, wherein the capsule includes a pressure sensor and activates compartments responsive to pressure levels inside the capsule.

11. The capsule of claim 1, wherein the capsule includes electrodes on a shell of the capsule and the electrodes are charged when delivering medication to induce electrophoresis.

12. The capsule of claim 1, wherein the one or more compartments are configured to be activated to sample or deliver multiple times.

13. The capsule of claim 1, wherein the one or more compartments include a piston to push content out of the compartment.

14. The capsule of claim 1, wherein the one or more compartments are initialized with a vacuum to pull in content for sampling.

15. A method of sampling the gastrointestinal tract or delivering content to the gastrointestinal tract, comprising:
ingesting a capsule with one or more compartments configured to sample the environment surrounding the capsule or configured to deliver content to the environment surrounding the capsule;
tracking the location of the capsule in real-time with a tracking system configured to identify the location of the capsule;
notifying an internal controller with the identified location of the capsule;
activating the one or more compartments to sample or deliver responsive to the location in the gastrointestinal tract.

16. The method of claim 15, wherein the internal controller activates compartments responsive to instructions from an external controller.

17. The method of claim 15, wherein the internal controller is configured to activate compartments responsive to sampling results received from another compartment that was previously activated.

18. The method of claim 15, wherein the internal controller activates multiple compartments simultaneously.

19. The method of claim 15, wherein the internal controller receives information from compartments that sample the environment relating to the type and/or count of bacteria surrounding the compartment.

20. The method of claim 15, wherein the capsule is protected by a shell with shell slots to serve as inlets or outlets for the capsule, wherein the compartments have compartment slots to serve as inlets or outlets from the compartments, and wherein the compartments are rotatable relative to the shell and are activated by aligning the compartment slot with the shell slot.
